(19) United States
(12) Patent Application Publication
(10) Pub. No.: US 2015/0064718 A1
(43) Pub. Date: Mar. 5, 2015

(54) ORGANIC/INORGANIC MAGNETIC COMPOSITE, METHODS OF MAKING AND USE THEREOF

(71) Applicant: Corning Incorporated, Corning, NY (US)

(72) Inventors: Stephen Joseph Caracci, Elmira, NY (US); Martin Joseph Murtagh, Trumansburg, NY (US); Christopher Bankole Shogbon, Corning, NY (US); Yue Zhou, Horseheads, NY (US)

(21) Appl. No.: 14/471,060

(22) Filed: Aug. 28, 2014

Related U.S. Application Data
(60) Provisional application No. 61/871,959, filed on Aug. 30, 2013.

(51) Int. Cl. G01N 33/543; B29C 47/00; B29C 47/40; H01F 1/01

(52) U.S. Cl. CPC G01N 33/5434; H01F 1/01; B29C 47/0004; B29C 47/0066; B29C 47/40; B29C 2793/009; B29K 2035/00
USPC 435/7.5; 252/62.54; 264/210.2; 428/402

(57) ABSTRACT

An organic/inorganic composite is provided. The organic/inorganic composite materials are sufficiently hard and brittle to be ground to form particles suitable for biological or chemical separation applications. The organic/inorganic composite materials can be magnetic or magnetically susceptible and can have functional reactive groups to allow attachment of biomolecules. Methods of making and using the organic/inorganic composites are also provided.

This application claims the benefit of priority under 35 U.S.C. §119 of U.S. Provisional Application Ser. No. 61/871,959 filed on Aug. 30, 2013 the content of which is relied upon and incorporated herein by reference in its entirety.

### TECHNICAL FIELD

The present disclosure relates to materials which combine organic thermoplastic material with magnetic inorganic material to form particles useful for affinity-binding applications, and methods of making and use thereof.

### BACKGROUND

Isolation and separation of biological materials, such as nucleic acids, proteins, viruses, cells and tissues, is a fundamental approach in biological research. Isolation provides a basis for characterizing biological materials, understanding structure and function, and for observing, cultivating, and conducting experiments and tests on biological systems.

One method for isolating and separating biological materials uses a solid bead coupled with a ligand having an affinity for a particular biological material. The solid bead comes in contact with a biological material in a mixture of material. The biological material binds to the affinity ligand, causing the biological material to bind to the bead. Then the solid bead can be separated from the mixture, accomplishing isolation of the biological material from the mixture.

An example of such a solid bead is a particle configured both to include an affinity ligand on its surface, for binding a biomolecule, and to be attracted to a magnetic field, for subsequent separation. Conventional methods of making such particles are based on graft, dispersion, or emulsion polymerization, coated on the surface of an inorganic magnetic core particle. The manufacture of these particles include multiple steps to coat a core magnetic particle with a polymer, chemically derivatize the polymeric surface, and then couple an affinity ligand to the surface. Conventional methods thus require numerous processing steps and result in liquid waste by-products that are detrimental to the environment. Thus, there is a need for organic/inorganic materials that can be used for isolation of biomolecules or biological systems that are easy to manufacture and/or environmentally friendly.

### SUMMARY

The disclosure provides, in embodiments, a method comprising extruding an organic/inorganic composite comprising a polymer and metal oxide through a twin screw extruder to form an extrudate, milling the extrudate to form organic/inorganic composite particles, wherein the metal oxide particles are dispersed throughout the composite. The milling step may comprise jet milling. In embodiments, the polymer may comprise a maleic anhydride moiety. In embodiments, the polymer may comprise styrene maleic anhydride copolymer (SMA) or polypropylene-graft-maleic anhydride (PPMA). In embodiments, the particles have an average diameter less than 10 μm. In embodiments, the particles have an average diameter less than 5 μm. In embodiments, the particles have an average diameter of between 1 μm and 10 μm. In embodiments, the particles have an average diameter of between 1 μm and 5 μm. In embodiments, the method further comprises conjugating one or more biomolecules to the composite. In additional embodiments, the disclosure provides an organic/inorganic composite comprising a thermoplastic polymer and iron oxide, wherein the iron oxide is dispersed throughout the polymer, wherein the composite has a Young's modulus greater than 1 gPa. In embodiments, the thermoplastic polymer comprises a maleic anhydride moiety. In embodiments, the organic/inorganic composite has a hardness of 0.08 gPa or greater. In embodiments, the organic/inorganic composite has a Young's modulus of between 1 gPa and 7 gPa. In embodiments, the composite has a Young's modulus greater than 1.5 gPa. In embodiments, the composite is in the form of particles less than 10 μm in average diameter. In embodiments, the composite is in the form of particles less than 5 μm in average diameter. In embodiments, the composite is in the form of particles between 1 μm and 10 μm in average diameter. In embodiments, the the composite is in the form of particles between 1 μm and 5 μm in average diameter. In embodiments, the particles further comprise one or more conjugated biomolecules. Additional embodiments will be disclosed and discussed below.

### BRIEF DESCRIPTION OF THE DRAWINGS

These and other aspects are better understood when the following detailed description is read with reference to the accompanying drawings.

FIGS. 1A, B, C and D are schematic illustrations of a manufacturing process, in an embodiment.

FIG. 2 is a schematic drawing of composite particles, such as those shown in the circle shown in FIG. 1.

FIG. 3 is a photomicrograph of an embodiment of organic/inorganic composite particles.

FIG. 4 is a graph illustrating particle size distribution of SMA organic/inorganic composite particles having different percentages of inorganic material.

FIG. 5 is a graph illustrating particle size distribution of PPMA organic/inorganic composite particles having different percentages of inorganic material.

FIG. 6 is a graph illustrating particle size distribution of PEMA organic/inorganic composite particles having different percentages of inorganic material.

FIG. 7 is a graph illustrating D50 vs. Young's modulus distribution of PEMA, SMA and PPMA organic/inorganic composite particles, illustrating the correlation between composite mechanical properties and particle sizes after grinding.

### DETAILED DESCRIPTION

Examples will now be described more fully hereinafter with reference to the accompanying drawings in which example embodiments are shown. Whenever possible, the same reference numerals and/or symbols are used throughout the drawings to refer to the same or like parts. However, aspects may be embodied in many different forms and should not be construed as limited to the embodiments set forth herein.

As set forth in the figures, example organic/inorganic composite materials and particles made from the materials are disclosed, including methods of making the materials and using the particles.

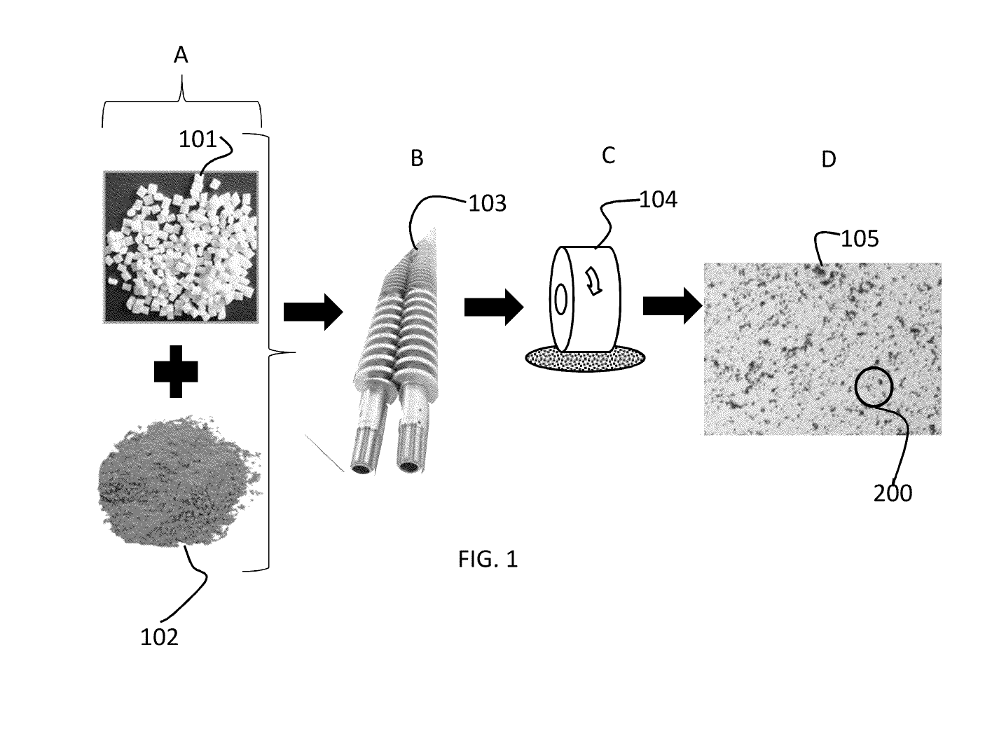

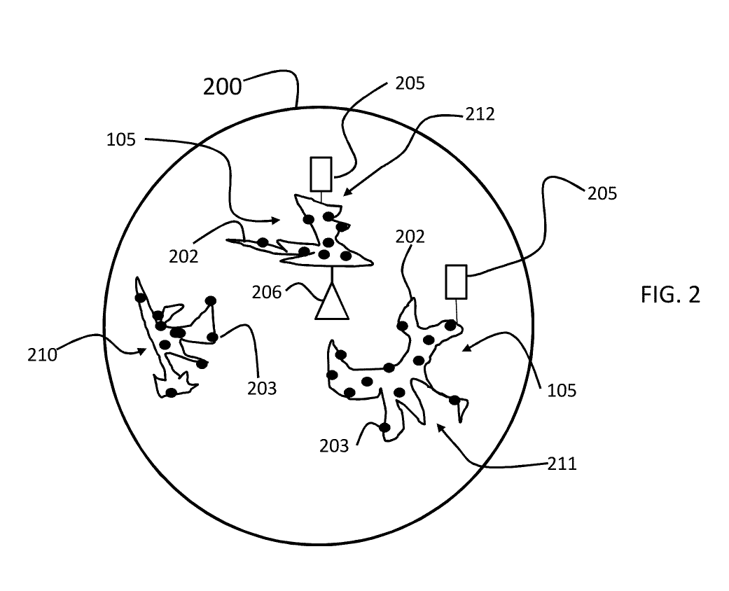

FIG. 1 provides a schematic illustration of a method of making an organic/inorganic composite material in an embodiment. As shown in step A, a polymer 101 is mixed with an inorganic material 102. In step B, the mixture is extruded through an extruder, for example a twin screw extruder 103. In step C, the extrudate is then ground into organic/inorganic composite particles 105. FIG. 1D is a photomicrograph of exemplary organic/inorganic composite particles 105. The circle 200 in FIG. 1, is shown, schematically, in FIG. 2.

A thermoplastic polymer can be provided in the form of resin, e.g. as pellets or in a similar form 101. The resin can be melted at a temperature suitable to convert the resin into a molten mass, e.g. at 250° C. for styrene maleic anhydride copolymer (SMA). Mixing can be carried out prior to and/or during the melting, with the mixing being done by various approaches, e.g. manually or mechanically, and to various extents, e.g. thoroughly in order to obtain homogeneous mixture. The thermoplastic solid phase can then be formed, for example, by melt spinning, melt extrusion, or injection molding, by use of an apparatus such as a spinneret, a twin screw extruder, a negative mold, or the like.

As shown in FIG. 1, a twin screw extruder 103 can be used. When a twin screw extruder is used, the extruder adds shear melting to the function of mixing the thermoplastic polymer with the inorganic material 102. The inorganic material that comprises the inorganic component of the organic/inorganic composite particles may be, for example, a magnetically susceptible particle. For example, a powder of the magnetically susceptible particle can be added before or during mixing. Similarly, an adduct can be added and mixed. A fiber, extruded from a twin screw extruder can be collected. The resulting fibers can then undergo further processing, such as grinding or milling 104 in order to create particles.

FIG. 2 is a schematic drawing of composite particles, such as those shown in the circle shown in FIG. 1. Organic/inorganic composite particles 105 are illustrated. The organic/inorganic composite particles 105 comprise an organic or thermoplastic polymer component 202, and an inorganic component 203. In embodiments, the thermoplastic polymer may have reactive functional groups. In addition, optionally, the functional groups of the polymers of the organic/inorganic composite particles 105 may be bound to conjugated to biomolecules 205, 206. Biomolecules may be molecules suitable for bio-extractions such as, for example, carboxyl groups, biotin, protein A, antibodies, affinity ligands or the like. The organic/inorganic composite particles 105 may be unmodified biomolecules in embodiments, as shown, for example at 210. Or, in embodiments, the organic/inorganic composite particles 105 may be modified to comprise a single type of biomolecule 205, as shown, for example at 211. Or, in embodiments, the organic/inorganic composite particles 105 may be modified to comprise multiple biomolecules 205, 206, as shown, for example at 212.

The methods of making organic/inorganic composite particles can also include a step of reacting reactive functional groups distributed on the surface of organic/inorganic composite particles, for example by simple mild acid or base hydrolysis of a cyclic anhydride or epoxide group, to present at the surface a derivative functional group. The derivative functional group can include, for example, a neutral or ionic form of a carboxylic acid group, an amine group, a hydroxyl group, a thiol group, a phosphate group, a sulfonate group, an ether group, an amide group, an amidate group, a sulfone group, a sulfoxide group, a disulfide group, or a thioether group.

In embodiments, the thermoplastic polymer comprises a reactive functional group. The term “reactive functional group”, as used herein, refers to a functional group that can react, in the presence of a suitable co-reactant, under suitably mild conditions, e.g. a pH that is neutral, mildly acidic, or mildly basic, and/or at ambient or slightly elevated temperature, but that nonetheless tends not to react to a substantial extent under standard conditions during melt processing of thermoplastic polymers due to the absence of a suitable co-reactant. A reactive functional group can be, for example, a cyclic anhydride group. A cyclic anhydride group has a structure including two acyl groups bound to the same oxygen atom and joined in a ring. A cyclic anhydride group can react in the presence of a nucleophile, e.g. a primary amine, under mild conditions, to yield a derivative product including an acyl group, e.g. an amide, and a carboxylic acid group. Yet cyclic anhydride groups tend not to react under standard conditions during melt processing of thermoplastic polymers, due to an absence of stoichiometric amounts of a suitable co-reactant. Exemplary cyclic anhydride groups include succinic anhydride and maleic anhydride.

A reactive functional group can also be, for example, an epoxide group. An epoxide group has a structure including a cyclic ether with three ring atoms. An epoxide group can react in the presence of a nucleophile, e.g. a primary amine, under mild conditions to yield a derivative product, i.e. a ring-opened nucleophilic addition product, yet epoxide groups also tend not to react under standard conditions during melt processing of thermoplastic polymers, again due to an absence of stoichiometric amounts of a suitable co-reactant.

Additional examples of reactive functional groups include neutral or ionic form of a carboxylic acid group (e.g. —COOH), an amine group (e.g. —NH2), a hydroxyl group (e.g. —OH), a thiol group (e.g. —SH), a phosphate group (e.g. —OP(OH)3), a sulfonate group (e.g. —SO2O—), an ether group (e.g. —C—O—C—), an amide group (e.g. RC(O)NR′2), an amidate group (e.g. R1NR2R3), a sulfone group (e.g. RS(O)2R′), a sulfoxide group (e.g. RS(O)R′), a disulfide group (e.g. —S—S—), or a thioether group (e.g. —C—S—C—). For example, by reacting a primary amine with a reactive functional group corresponding to cyclic anhydride, a derivative product can be obtained including an acyl group, i.e. an amide of the primary amine, and a carboxylic acid group, i.e. an exemplary derivative functional group. As noted above, this reaction can be carried out under mild conditions. In another example, a reactive functional group corresponding to a cyclic anhydride or an epoxide group can be hydrolyzed under mild acid or base conditions to open the anhydride or epoxide, respectively, to yield carboxylic acid derivative functional groups or hydroxyl derivative functional groups, respectively. Numerous variations will also be apparent.

In embodiments, reactive functional groups are part of the thermoplastic polymer. The reactive functional groups can be a polymer backbone group or a polymer pendant group. A moiety being a polymer backbone group can be, for example, a cyclic anhydride group or an epoxide group that is included in the chain of atoms that forms the backbone of a molecule of the thermoplastic polymer. A moiety being a polymer pendant group can be, for example, a cyclic anhydride group or an epoxide group that is part of a side group of a molecule of the thermoplastic polymer. Reactive functional groups can be distributed on a surface of organic/inorganic composite or composite particles. For example, reactive functional groups can be present on part or all of the surface and/or can extend outward from the surface. The distribution of reactive functional groups can be homogeneous with respect to part or all of the surface, e.g. uniform, regular, even, and/or not in a gradient along or across the surface. The distribution can also be heterogeneous with respect to part or all of the surface, e.g. non-uniform, irregular, uneven, and/or in a gradient along or across the surface. In addition, the moieties may be absent from part of the surface of the organic/inorganic composite or composite particles. Thus, for example, the distribution can be homogeneous or heterogenous with respect to the entire surface of the organic/inorganic composite or composite particles. The distribution can also be homogeneous with respect to one or more parts of the surface and heterogeneous with respect to another part or parts, with the reactive functional groups being absent from any part or parts that may remain. Also for example, the organic/inorganic composite or composite particles can include more than one surface, e.g. multiple distinct surfaces and/or complex irregular surfaces, depending on the shape and/or porosity of the organic/inorganic composite or composite particles, with the reactive functional group being distributed on one or more of these surfaces as described above.

The reactive functional group can be distributed homogeneously within the organic/inorganic composite or composite particles. For example, the reactive functional groups may be distributed, throughout the solid phase uniformly, regularly, evenly, in a manner indicative of thorough mixing during melt processing, and/or not in a gradient. The distribution can be homogeneous even if the solid phase includes some local or micro-heterogeneities. For example, although the presence of a magnetically susceptible particle and/or an adduct in the solid phase may create local or micro-heterogeneities with respect to the structure of the organic/inorganic composite or composite particles.

The reactive functional groups, including those distributed on the surface of the organic/inorganic composite or composite particles can be derivatized to covalently attach a biomolecule 205, 206, as shown in FIG. 2. As noted above, the biomolecule can be a ligand, or an affinity ligand, e.g. one or more moieties and/or molecules that can selectively bind a biomolecule based, for example, on non-covalent interactions and/or a complementary fit between a ligand and a biomolecule. The biomolecule can be any molecule that binds to, is specific to, diagnostic of, derived from, or otherwise characteristic of at a chemical interaction or reaction, a cell-cell interaction, or an interaction on a tissue or organism scale. An affinity ligand can be specific to a single biomolecule, a group of biomolecules, or may be general to a class of biomolecules. An affinity ligand, or a mixture of affinity ligands, can be used to target single biomolecule or a class of biomolecules. An affinity ligand can also be specific to a biomolecule that is associated with a larger structure, including for example a complex of proteins, nucleic acids, and/or other compounds, or a biological system, such as a virus or cell.

An affinity ligand can be, for example, a peptide, a protein, an oligonucleotide, a gene, an extracellular matrix peptide or preparation, a polymer, a polylysine, a polyphosphate, polyethylene glycol, a polyethylene glycol amine, streptavidin, biotin, a lipid, a cell extract, or a basement membrane preparation.

The biomolecule can be, for example, a protein, a glycoprotein, a lipid, a phospholipid, a polysaccharide, a nucleic acid polymer, an oligonucleotide, a cell-surface component, a cell-membrane component, a cell-wall component, a viral-surface component, a viral-envelope component, or a viral-capsid component.

The reactive functional group of the polymer can be derivatized to bind a specific biomolecule. For example, polymer having a maleic anhydride moiety, mixed with an iron oxide inorganic material, extruded and milled to form particles, can be derivitized to form carboxyl groups on the surface of the particles, which bind DNA. These particles, magnetized, can be used to remove DNA from solution. For example these particles can be used to clean up the results of PCR assays.

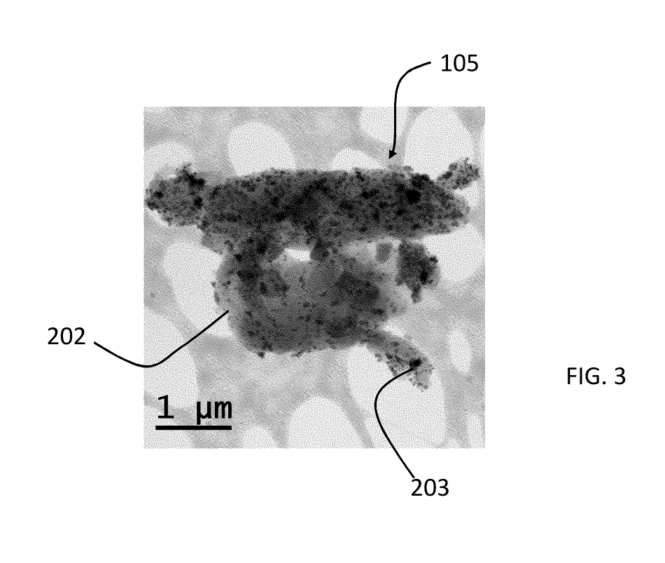

FIG. 3 is a photomicrograph of an embodiment of organic/inorganic composite particles 105. FIG. 3 shows the polymer 202 portion of the particle 105, and the metal oxide cluster component 203 of the particle, distributed throughout the particles 105.

Importantly, it has been found that in order to form particles useful in separation applications, the organic/inorganic composite material formed using the methods shown in FIG. 1, must have certain characteristics. That is, the combination of organic material and inorganic material must have a certain brittleness and strength, or Young's modulus, to allow the extruded material to form particles of an appropriate size when milled. Polymers that are too soft and malleable do not form particles, but instead form a gummy material.

Often, in the prior art, malleability was a desirable feature in composite organic/inorganic materials. For example, US Patent publication no. 2013/0130026 discloses a polymer composite having magnetic properties having improved malleability, ductility, viscoelastic and thermoplastic properties. In contrast, in the present application, in the organic/inorganic composite material described herein, brittleness is a desired feature of the material. A more brittle material will fracture during milling to form particles of an appropriate size to perform for separation applications.

It is believed that the brittleness of the organic/inorganic composite material results from both particle deformation and fracture mechanisms of the material. The material must break to form particles of a suitable size distribution for use in separation applications during milling. The particle fracture behavior of a material in general depends on the mechanical properties of the material to be milled, processing conditions like stressing intensity, impact velocity, temperature, and the pre-existing imperfections and flaws in the material.

To understand the relationship between measurable organic/inorganic composite material characteristics and materials that resulted in suitable particles for separation applications, a series of polymers having maleic anhydride groups and different mechanical properties were compounded with iron oxide at different concentrations using a twin screw extruder. Characteristics such as hardness or fracture touchness and Young's modulus or elastic modulus of the extruded organic/inorganic composite materials were measured. The composite materials were then subjected to totar mill and jet mill processes and particle sizes were measured. The relationship between mechanical properties and size of the particles after milling were analyzed. Table 1 reports hardness, Young's modulus, the ratio of hardness to Young's modulus and particle size for several materials, in embodiments. The polymers included in this study include (1) Poly(styrene-co-maleic anhydride) (SMA). (2) Polypropylene-graft-maleic anhydride (PPMA); and (3) Poly(ethylene-co-glycidyl methacrylate) (PEMA). Compositions of the iron oxide with each polymer are: (1) SMA with 0%, 5%, 10%, 20% or 30% iron oxide; (2) PPMA with 0%, 10%, 30%, 60% iron oxide; and (3) PEMA with 0%, 10%, 30%, 60% iron oxide.

After compounding, small quantities of the composites were compressed in the form of thin sheets about 1 mm in thickness, and about 20 mm in the other two dimensions, which were submitted for nano-indentation. Young's modulus and hardness were obtained from these materials. The composites were then subjected to the jet milling process. The size distributions of obtained particles were characterized. Then the particles were hydrolyzed using pH 9 boric buffer to convert the anhydride groups to carboxyl groups.

TABLE 1

| | | Young's modulus | Young's modulus/ | Particle Size |
|---|---|---|---|---|
| | Hardness (gPa) | (gPa) | hardness | D50 (μm) |
| SMA 0% Iron Oxide | 0.26 ± 0.02 | 4.76 ± 0.29 | 18.2 | 4.01 |
| SMA 10% Iron Oxide | 0.23 ± 0.05 | 3.34 ± 1.12 | 14.2 | 4.82 |
| SMA 30% Iron Oxide | 0.26 ± 0.07 | 5.27 ± 1.03 | 20.1 | 2.99 |
| PPMA 0% Iron Oxide | 0.09 ± 0.07 | 2.01 ± 1.20 | 21.8 | 3.93 |
| PPMA 30% Iron Oxide | 0.19 ± 0.03 | 3.84 ± 0.71 | 20.4 | 3.32 |
| PPMA 60% Iron Oxide | 0.25 ± 0.19 | 5.27 ± 1.80 | 21.3 | 2.87 |
| PEMA 0% Iron Oxide | 0.026 ± 0.006 | 0.42 ± 0.15 | 16.2 | N/A |
| PEMA 30% Iron Oxide | 0.016 ± 0.012 | 0.23 ± 0.14 | 14.1 | 17.22 |
| PEMA 60% Iron Oxide | 0.037 ± 0.017 | 0.7 ± 0.16 | 19.0 | 16.56 |

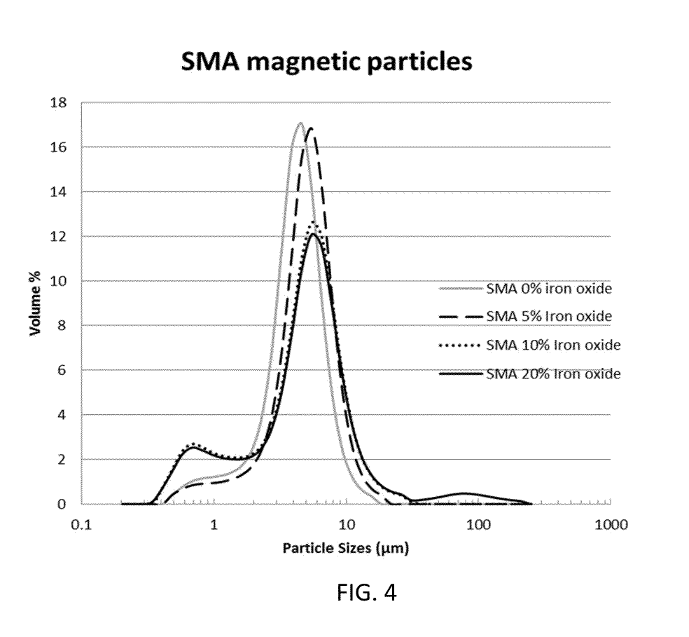

FIG. 4 is a graph illustrating particle size distribution of SMA organic/inorganic composite particles having different percentages of inorganic material. FIG. 4 illustrates that SMA organic/inorganic composite materials formed particles with average sizes less than 10 μm, with a tight distribution of particle sizes, regardless of the concentration of iron oxide in the mixture. Table 1 reports SMA organic/inorganic composite materials having varying percentages of iron oxide. These materials, prepared as discussed above, were measured for hardness, Young's modulus, Young's modulus/hardness and particle size. SMA material formed particles that were very hard, with very high Young's modulus, at least higher than 1 gPa. These materials can also be described as having a hardness of 0.08 gPa or greater, having a Young's modulus of between 1 gPa and 7 gPa or having a Young's modulus greater than 1.5 gPa. SMA material formed particles less than 10 μm in average diameter. SMA particles can also be described as having particle size less than 5 μm in average diameter, forming particles between 1 μm and 10 μm in average diameter or forming particles between 1 μm and 5 μm in average diameter.

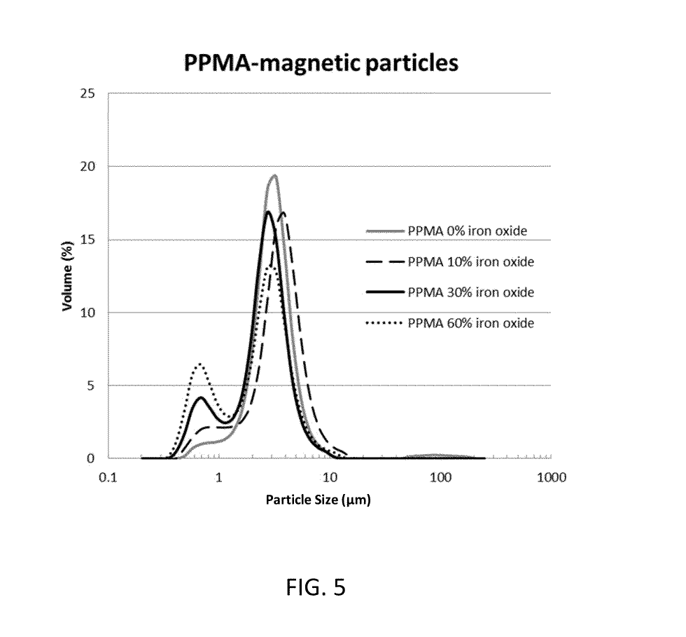

FIG. 5 is a graph illustrating particle size distribution of PPMA organic/inorganic composite particles having different percentages of inorganic material. FIG. 5 illustrates that PPMA organic/inorganic composite materials formed particles with average sizes less than 10 μm, with a tight distribution of particle sizes, regardless of the concentration of iron oxide in the mixture. These materials, prepared as discussed above, were measured for hardness, Young's modulus, Young's modulus/hardness and particle size and the results are reported in Table 1. Table 1 reports PPMA organic/inorganic composite materials as being very hard, with very high Young's modulus, at least higher than 1 gPa. Young's modulus of these materials can also be described as having a hardness of 0.08 gPa or greater, having a Young's modulus of between 1 gPa and 7 gPa or having a Young's modulus greater than 1.5 gPa. PPMA materials formed particles less than 10 μm in average diameter. The particle size of PPMA materials can also be described as being particles less than 5 μm in average diameter, particles between 1 μm and 10 μm in average diameter, or particles between 1 μm and 5 μm in average diameter.

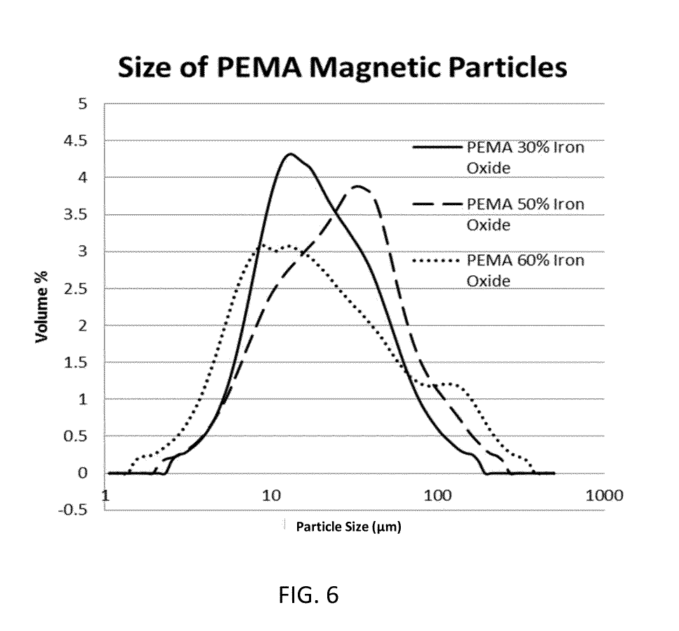

FIG. 6 is a graph illustrating particle size distribution of PEMA organic/inorganic composite particles having different percentages of inorganic material. FIG. 6 illustrates that PEMA organic/inorganic composite materials formed particles with average sizes greater than 10 μm, with a loose distribution of particle sizes. These materials, prepared as discussed above, were measured for hardness, Young's modulus, Young's modulus/hardness and particle size and the results are reported in Table 1. Table 1 reports PEMA organic/inorganic composite materials as less hard than materials formed from SMA or PPMA. PEMA organic/inorganic composite materials had lower high Young's modulus and formed particles greater than 10 μm in average diameter. In some cases PEMA processessing did not result in particles. Gummy material was formed, even after long periods of milling. (in Table 1, n/a is entered where no particles were formed).

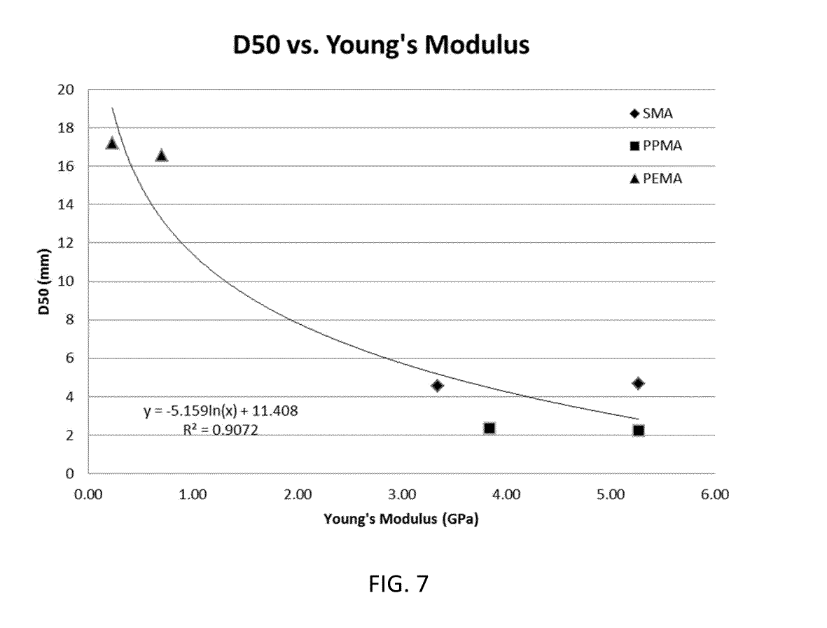

FIG. 7 is a graph illustrating D50 particle size distribution vs. Young's modulus distribution of PEMA, SMA and PPMA organic/inorganic composite particles, illustrating the correlation between composite mechanical properties and particle sizes after grinding. FIG. 7 illustrates that materials having a Young's modulus greater than 1 GPa, such as SMA and PPMA, but not PEMA, are suitable for forming organic/inorganic composite particles in embodiments.

Exemplary copolymers of maleic anhydride or glycidyl methacrylate that are commercially available include the following: methyl vinyl ether/maleic anhydride copolymer; polyethylene maleic anhydride copolymer; poly(ethylene-co-ethyl acrylate-co-maleic anhydride); poly(isobutylene-alt-maleic anhydride); vinyl acetate-maleic anhydride copolymer; polypropylene-graft-maleic anhydride; poly(maleic anhydride-alt-1-octadecene); poly(vinyl chloride/vinyl acetate/maleic acid) 86:13:1; 2,5-furandione, polymer with ethene and 1-propene; poly(butadiene/maleic anhydride) [Sartomer Ricobond]; 2,5-furandione, polymer with 1,3-butadiene and ethenylbenzene [Sartomer Rincon]; poly(methyl vinyl ether-alt-maleic anhydride), cross-linked with 1,9-decadiene; octadecyl vinyl ether-maleic anhydride copolymer, poly(ethylene-co-glycidyl methacrylate); poly(ethylene-co-methyl acrylate-co-glycidyl methacrylate [glycidyl methacrylate, 8 wt. %, methyl acrylate 25 wt. %]; 2-propenoic acid, 2-methyl-, oxiranylmethyl ester, polymer with ethene and ethenyl acetate; 2-propenoic acid, 2-methyl-, polymer with ethenylbenzene and oxiranylmethyl 2-methyl-2-propenoate; and poly(tert-butyl methacrylate-co-glycidyl methacrylate), as listed in TABLE 2.

The organic/inorganic composite particles may include one or more of the thermoplastic polymers. Thus, for example, the thermoplastic solid phase may include only one of the above-noted polymers. Also for example, the organic/inorganic composite particles may include a blend of two or more of the above-noted polymers.

As discussed briefly above, the inorganic portion of the organic/inorganic composite particles can be a magnetically susceptible particle. A magnetically susceptible particle refers to a particle that can be attracted to a magnetic field, e.g. an externally applied magnetic field. The magnetically susceptible particle can be made of a metal, a metal oxide (e.g. an iron oxide), certain types of ceramics, or another substance that can be attracted to a magnetic field. The magnetically susceptible particle can be of a sufficiently small size such that the particle is not itself permanently magnetized, avoiding self-attraction and/or self-aggregation of the particle (e.g. of powders thereof) in the absence of an externally applied magnetic field. The magnetically susceptible particle can be, for example, a paramagnetic particle, a superparamagnetic particle, an inorganic oxide particle, an iron oxide particle, a cobalt iron oxide particle, a zinc iron oxide particle, a cobalt-zinc iron oxide particle, a nickel iron oxide particle, a nano-sized magnetite particle, a non-oxide particle, or an iron platinum particle. The magnetically susceptible particle can be included in an amount sufficient to impart magnetic susceptibility to the affinity-binding solid phase, e.g. based on about 1 to about 50 w/w % loading of a powder of the magnetically susceptible particle (i.e. adding the magnetically susceptible particle to 1 to 50 w/w % of the final mixture for melt processing), or 5 to 40 w/w % loading, or 15 to 25 w/w % loading. As will be appreciated, the same loadings can have lower values when expressed in terms of v/v %, based on the magnetically susceptible particle having a higher density than the thermoplastic polymer. For example, for an oxide particle having a density that is five-fold greater than that of a thermoplastic polymer, a loading of 50 w/w % would be approximately equal to a loading of about 17 v/v %. As noted above, if the magnetically susceptible particle is reactive, e.g. based on having had its surface chemically modified with a reactive moiety, then it should not be added in an excessive amount, e.g. in an amount that could cause extensive cross-linking Like the reactive functional groups, the magnetically susceptible particle (e.g. a powder thereof) can be distributed homogeneously within the solid phase. The organic/inorganic composite particles can be a nanocomposite based on the presence of the magnetically susceptible particle.

When the thermoplastic is melt blended with the inorganic field responsive nanoparticles (the iron oxide), the formed mixture cools to a brittle fracturable solid mass. This solid mass allows for particalization down to the micron and sub-micron scale. For example, the melt-blended mixture containing a 50/50 wt % of thermoplastic to iron oxide when melted together will yield a solid mass that can be pulverized or jet-milled or wet-milled down to particles whose distributions average in the 3 micron, 2 micron, 1 micron, or 0.5 micron range. This material is useful in that it can be manufactured easily and inexpensively without the need for expensive processing. Or, these thermoplastic/field responsive nanoparticle mixtures can be cryomilled to form these particles. However, a cryomilling process is very expensive and requires significant temperature control.

The organic/inorganic composite particles can further include an adduct. The adduct can be an additive or filler that is present in the organic/inorganic composite based on having been added during melt processing. The adduct can be added, for example, to strengthen the organic/inorganic composite material without relying on cross-linking of the thermoplastic polymers thereof, to aid in visualization or detection of the material, to provide the material with a porous structure following melt processing, and/or to improve mechanical properties of the material. The adduct can be, for example, a carbon nanotube, graphene, a silica bead, a coloring agent, a fluorescent agent, a quantum dot, a zeolite, a salt, an organic filler, a non-reactive polymer filler, polyethylene, poly(methyl methacrylate), polystyrene, an inorganic filler, a nonmagnetic inorganic filler, a fluorescently doped silica nanoparticle, a glass, a glass particle, or a glass fiber. Like the reactive functional groups, the adduct can be distributed homogeneously within the solid phase. The organic/inorganic composite material can be a nanocomposite based on the presence of the adduct therein.

The organic/inorganic composite particles can form at least a portion of an article of manufacture. The article of manufacture can be, for example, a plate, a tube, a slide, a bottle, a fiber, a chopped fiber, a particle, a bead, a powder, a microcarrier, or a scaffold. Articles of these types are well known in the field, and include for example microtiter plates including wells, microcentrifuge tubes, and microscope slides, each of which are used in a variety of analytical and clinical applications. Thus, for example, organic/inorganic composite particles can form part of such an article, e.g. the well portions of a microtiter plate, or the whole article, e.g. a fiber, chopped fiber, particle, or bead.

The organic/inorganic composite particles can be used in various methods, for example for isolation, purification, and/or quantitation of biomolecules. An example method of use can include a step of contacting a substance including a biomolecule with the organic/inorganic composite particles such that an affinity ligand bound to the organic/inorganic composite particles binds a biomolecule.

The contacting step can be carried out in various ways, depending on the form of the organic/inorganic composite particles and the substance including the biomolecule. For example, for organic/inorganic composite particles in the form of a plate, tube, slide, or bottle, and a substance including the biomolecule in the form of a solution, the substance solution can be transferred to a surface of the organic/inorganic composite particles, e.g. by pipetting, pouring, or another transfer processes. Also for example, for organic/inorganic composite particles in the form of a fiber, particle, bead, or powder, and a substance including the biomolecule in the form of a solution, the organic/inorganic composite particles can be added to the substance, e.g. by submerging the organic/inorganic composite particles therein.

The contacting step can also be carried out under conditions that promote binding of an affinity ligand and a biomolecule, such that the biomolecule becomes immobilized on the organic/inorganic composite particles. Such conditions can be determined readily based on principles of chemistry, and numerous conditions suitable for particular combinations of affinity-ligands and biomolecules are well known.

The method can also include a step of isolating the biomolecule from the substance. The separation can be accomplished, for example, by fluidic flow of a mobile liquid or solvent phase of the substance across or between surfaces of the organic/inorganic composite particles, e.g. by column chromatography separation, apheresis, or centrifugal spin column separation. Other approaches, such as physically withdrawing the organic/inorganic composite particles from the substance, e.g. by pulling a organic/inorganic composite particle from a solution of the substance, or magnetic separation, e.g. by magnetic separation of affinity-bound thermoplastic organic/inorganic composite particles that also includes a magnetically susceptible particle, can also be used.

Thus, for example, in some embodiments the affinity ligand can be used to bind a corresponding biomolecule with sufficient selectivity for isolation of the biomolecule from a substance including at least one undesired component. Also for example, in some embodiments the affinity ligand can be used for isolation of an undesired biomolecule from a substance including at least one desired target component. Also for example, in some embodiments the affinity ligand can be used for isolation of complexes of protein, nucleic acid, and/or other compounds, or biological systems, such as cells or viruses, based on association of the corresponding biomolecule with the complexes or biological systems, e.g. by non-covalent interactions and/or covalent binding.

The magnetically susceptible organic/inorganic composite particles also can be used in methods for isolation and/or purification of biomolecules. An example method of use can include contacting a substance including a biomolecule with the solid phase such that organic/inorganic composite particles binds the biomolecule through a derivative reactive functional group. The binding through the derivative functional group can be direct, such that the biomolecule binds the derivative functional group itself. The binding can also be indirect, for example where the derivative functional group has been further derivatized based on covalent attachment of an affinity ligand for the biomolecule, such that the biomolecule binds the affinity ligand.

In an aspect (1), the disclosure provides a method comprising: extruding a composition comprising a polymer and metal oxide through a twin screw extruder to form an extrudate; milling the extrudate to form organic/inorganic composite particles, wherein the metal oxide particles are dispersed throughout the organic/inorganic composite particles.

In an aspect (2), the disclosure provides the the method according to aspect 1, wherein the milling step comprises jet milling.

In an aspect (3), the disclosure provides the method according to aspect 1 or 2 wherein the polymer comprises a reactive functional group.

In an aspect (4), the disclosure provides the method according to aspect 3 wherein the reactive functional group is a maleic anhydride group.

In an aspect (5), the disclosure provides the method according to aspect 3 or 4 wherein the polymer comprises styrene maleic anhydride copolymer (SMA) or polypropylene-graft-maleic anhydride (PPMA).

In an aspect (6), the disclosure provides the method according to any one of the preceeding aspects wherein the metal oxide is iron oxide.

In an aspect (7), the disclosure provides the method according to any one of aspects 1-6, wherein the particles have an average diameter less than 10 μm.

In an aspect (8), the disclosure provides the method according to any one of aspects 1-6, wherein the particles have an average diameter less than 5 μm.

In an aspect (9), the disclosure provides the method according to any one of aspects 1-6, wherein the particles have an average diameter of between 1 μm and 10 μm.

In an aspect (10), the disclosure provides the method according to any one of aspects 1-5, wherein the particles have an average diameter of between 1 μm and 5 μm.

In an aspect (11), the disclosure provides the method according to any one of the preceeding aspects, further comprising conjugating a biomolecule to the organic/inorganic composite particles.

In an aspect (12), the disclosure provides the method according to any one of the preceeding aspects, further comprising conjugating a plurality of biomolecules to the organic/inorganic composite particles.

In an aspect (13), the disclosure provides an organic/inorganic composite comprising a thermoplastic polymer comprising a maleic anhydride group and iron oxide, wherein the iron oxide is dispersed throughout the polymer, wherein the composite has a Young's modulus greater than 1 gPa.

In an aspect (14), the disclosure provides an organic/inorganic composite of aspect 13 wherein the composite has a hardness of 0.08 gPa or greater.

In an aspect (15), the disclosure provides an organic/inorganic composite of aspect 13 or 14 wherein the composite comprises a Young's modulus of between 1 gPa and 7 gPa.

In an aspect (16), the disclosure provides an organic/inorganic composite of any one of aspect 13-15 wherein the composite comprises a Young's modulus greater than 1.5 gPa.

In an aspect (17), the disclosure provides an organic/inorganic composite of any one of aspect 13-15, wherein the composite is in the form of particles less than 10 μm in average diameter.

In an aspect (18), the disclosure provides an organic/inorganic composite of any one of aspect 13-15 wherein the composite is in the form of particles less than 5 μm in average diameter.

In an aspect (19), the disclosure provides an organic/inorganic composite of any one of aspect 13-15, wherein the composite is in the form of particles between 1 μm and 10 μm in average diameter.

In an aspect (20), the disclosure provides an organic/inorganic composite of any one of aspect 13-15, wherein the composite is in the form of particles between 1 μm and 5 μm in average diameter.

In an aspect (21), the disclosure provides an organic/inorganic composite of any one of aspect 13-20 further comprising conjugating a biomolecule to the organic/inorganic composite particles.

In an aspect (22), the disclosure provides an organic/inorganic composite of any one of aspect 13-21 further comprising conjugating a plurality of biomolecules to the organic/inorganic composite particles.

### EXAMPLES

### Example 1

### Preparation of Magnetically Susceptible SMA-Magnetite Particles by Hand

The styrene maleic anhydride (“SMA”) copolymer XIRAN™ from Polyscope Polymers B.V. (SZ 15170 (lot #101215-A5)) with a melt flow index (MFI) of 2.7 grams at 230° C./2.16 kg, was dry blended with magnetite iron oxide particles (20-30 nm) from Nanostructures and Amorphous materials Inc. (Stock #2540TR, CAS 1309-37-1) in an 80:20 percent by weight mixture using a vortex shaker. For manual mixing, the dry powder mixture was combined in an Erlenmeyer flask and heated with manual stirring using a metal stirring rod or spatula on a hot plate set to ˜250° C. After about 20 minutes of manual mixing the dry powder formed a solid molten mass of reddish brown color. Alternatively, mechanical mixing in a Brabender feed mixer can be used for larger scale mixing. Upon cooling, the resulting solid mixture of SMA-magnetite blend was scraped out of the flask bottom and placed into a mortar and pestle for manual grinding. Alternatively, the SMA-magnetite blend can be particle formed through a range of grinding methods including ball milling and jet milled micronizers. Prior to particalization the material can be first made from an extruded fiber with a defined diameter and then ground as a feed material for jet milling. The manually ground particles were >10 microns in size while the ball milled particles were ˜1 to 10 microns in size.

### Example 2

### Preparation of Magnetically Susceptible SMA-Magnetite Particles by Use of a Brabender Feed Mixer and Roller Bottle Grinding

The styrene maleic anhydride copolymer XIRAN™ and magnetite iron oxide particles from the same lots as described above were used to make three 250 g batches as follows: (i) 10% loading included 225 g SMA polymer and 25 g magnetite particles, (ii) 20% loading included 200 g SMA polymer and 50 g magnetite particles, and (iii) 40% loading included 150 g SMA polymer and 100 g magnetite particles. Batch sizes of 250 g were used to ensure sufficient material for consistent mixing by the Brabender feed mixer. The mixer was heated to an internal temp of 250° C. for the entire process. Components were first weighed and dry blended according to the 250 g batch amounts described above. For each batch, the mixture was then added into the Brabender feed mixer and allowed to sit for approximately 2 minutes for melting to begin. The mixer was then turned on with a forward RPM of 50 and mixing was conducted for 5 minutes. Following the mixing, the front of the mixer was removed. Then the resulting SMA-magnetite blend was scraped out with a spatula, allowed to cool on foil, and bagged and labeled.

Large pieces of the SMA-magnetite blend as prepared by use of the Brabender mixer were reduced to small pieces either by wrapping the large pieces in a clean cloth and then striking with a hammer or by grinding the large pieces with a hand held coffee grinder. The resulting small pieces of the SMA-magnetite blend were then placed in a high-density-polyethylene (HDPE) bottle, along with zirconia grinding media of about a half an inch in diameter. The bottle was then closed, placed on its side in a roller mill, and rotated at about 100 RPM for 3 days, resulting in grinding of the small pieces of the SMA-magnetite blend to a powder. Particles of the SMA-magnetite blend were then isolated by addition of hexanes to form a slurry, followed by collection of particles from the slurry by use of a Buchner funnel with vacuum filtration.

### Example 3

### Biotin Conjugation to Magnetically Susceptible SMA-Magnetite Particles

Particles of the SMA-magnetite blend prepared by manual grinding as described above were tested for preservation of amine surface reactivity by biotin immobilization. Approximately 300 mg of particles of the SMA-magnetite blend were added to a glass vial fitted with a small TEFLON® coated magnetic spin bar. A 3 ml solution of 50 μM biotin-PEG₂-amine (Thermo Scientific, EZ Link® cat #21346, MW. 374.51 g/mole) in 100 mM borate pH 9.0 solution was then added to the vial, resulting in a particle concentration of 100 mg/ml. Then the biotin-PEG₂-amine was allowed to react for 1 hour with magnetic stirring, resulting in a biotin coating of the SMA-magnetite particles. Following the biotin immobilization, the particles were captured magnetically using a solid state neodymium magnet. The magnetically captured particles were washed with distilled deionized water several times using aspiration and magnetic capture. Then the particles were finally completely surface deactivated, i.e. “blocked,” by applying a 100 mM TRIS pH 8.0 buffer for 30 minutes.

In order to validate the presence of biotin on the biotin-coated SMA-magnetite particles, a streptavidin binding assay was performed using two different colorimetric enzyme detection systems

First, a peroxidase conjugated streptavidin (“streptavidin-HRP”) reagent (Jackson immuno Research Labs, 016-160-084) solution was prepared at 10 μM from a stock vial of 1.53 mg/ml. A 50 μL solution of the 100 mg/ml biotin-coated SMA-magnetite particles was combined with a 50 μL solution of 10 μM streptavidin-HRP in a 96 microwell CELLBIND® plate (Corning). The binding was allowed to proceed for 30 minutes. Then the particles were magnetically captured using a 96 well microplate magnet. The streptavidin-HRP solution was removed and replaced with 1×PBS/0.01% Tween wash solution. Following three washes the particles were again captured. A detecting solution of SUREBLUE RESERVE™ TMB microwell peroxidase solution (KPL Labs, 53-00-02) was then applied. After a few seconds an intense saturating blue color was clearly evident, strongly indicating the presence of the biotin on the SMA-magnetite particles, thus confirming biotin coating of the particles.

Second, an alkaline phosphatase-conjugated streptavidin (“AP-SA”) reagent (Jackson immuno Research Labs, 016-050-084) was used in the same amounts and concentrations as described above for streptavidin-HRP except that the colorimetric system was FIREPHOS™ microwell phosphatase solution (KPL Labs, 50-87-20). As in the case of the streptavidin-HRP system the alkaline phosphatase system also developed a strong colorimetric signal rapidly, this time in bright red color. An alkaline phosphatase enzyme stopping solution APSTOP™ (KPL Labs, 50-89-00) was used to control the AP-SA generated color after 10 minutes of signal generation, further confirming biotin coating of the particles.

The biotin-coated SMA-magnetite particles alone, not exposed to any enzyme system, yielded no signal in either signaling system, confirming that the signals generated during the treatments with streptavidin-HRP and AP-SA were based on streptavidin. Collectively, these results validate that the SMA polymer retains its amine surface reactivity after formation of the melt blended magnetite iron oxide composite.

### Example 4

### Biotin Conjugation to SMA Injection Molded Slides

An injection molded slide made from the styrene maleic anhydride copolymer XIRAN™ as described above was tested for preservation of amine surface reactivity by a series dilution of biotin immobilization and use of Cy-3 labeled streptavidin conjugation to the biotin. The series dilution was carried out with (1) 0.5 mM biotin PEG amine, (2) 0.25 mM biotin PEG amine, and (3) 0.125 mM biotin PEG amine, followed by Tris blocking for surface deactivation, and then staining with Cy-3-labeled streptavidin at 1 μg/ml (0.017 μM). As shown in FIG. 6, signal was observed for each dilution. Moreover, it was observed that the Cy-3-labeled streptavidin self-quenched at concentrations above 0.25 μM. These results indicate that a significant amount of biotin was covalently bound to the surface of the SMA polymer injection molded slides and was viable for binding streptavidin. It will be apparent to those skilled in the art that various modifications and variations can be made without departing from the spirit and scope of the claimed invention.

TABLE 2
Commercially available copolymers of maleic anhydride or glycidyl methacrylate.

| CAS Number | Name | Structure | Melt index |
|---|---|---|---|
| 9011-13-6 | Styrene maleic anhydride (SMA) | 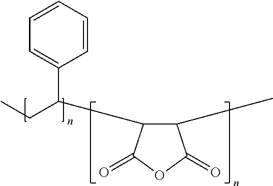 | |
| 9011-16-9 | Methyl vinyl ethyl/maleic anhydride copolymer | 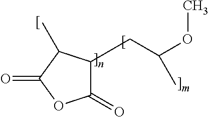 | |
| 9006-26-2 | Polyethylene maleic anhydride copolymer (PEMA) | 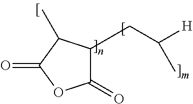 | |
| 41171-14-6 | Poly(ethylene-co-ethyl acrylate-co-maleic anhydride) | 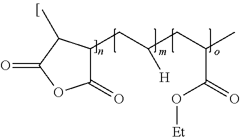 | melt index 7 g/10 min (190° C./2.16 kg) |
| 26426-80-2 | Poly(isobutylene-alt- maleic anhydride) | 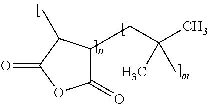 | |
| 9011-07-8 | Vinyl Acetate-Maleic Anhydride Copolymer | 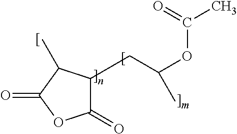 | |
| 25722-45-6 | Polypropylene-graft- maleic anhydride (PPMA) | 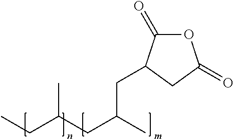 | |
| 25266-02-8 | Poly(maleic anhydride-alt-1- octadecene) | 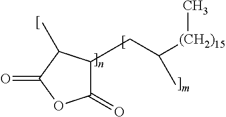 | |
| 25085-82-9 | Poly(vinyl chloride/vinyl acetate/maleic acid) 86:13:1 | 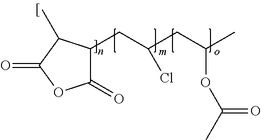 | |
| 31069-12-2 | 2,5-Furandione, polymer with ethene and 1-propene | 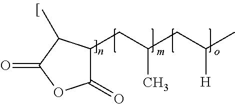 | |
| 25655-35-0 | Poly(butadiene/maleic anhydride) [Sartomer Ricobond] | 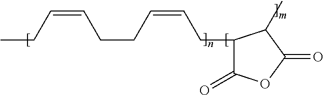 [SMILES: CC/C=C\CC/C=C\CC1C(=O)OC(=O)C1C] | |
| 27288-99-9 | 2,5-Furandione, polymer with 1,3- butadiene and ethenylbenzene [Sartomer Rincon] | 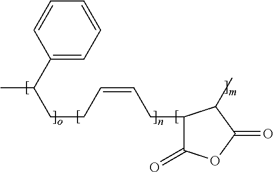 [SMILES: CC(CC/C=C\CC1C(=O)OC(=O)C1C)c1ccccc1] | |
| 136392-67-1 | Poly(methyl vinyl ether-alt-maleic anhydride), cross- linked with 1,9- decadiene | 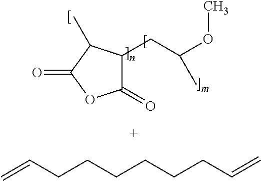 | |
| 28214-64-4 | Octadecyl vinyl ether- maleic anhydride copolymer | 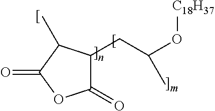 | |
| 26061-90-5 | Poly(ethylene-co- glycidyl methacrylate) | 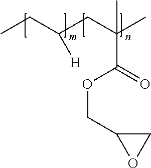 | melt index 5 g/10 min (190° C./ 2.16 kg) |
| 51541-08-3 | Poly(ethylene-co- methyl acrylate-co- glycidyl methacrylate (glycidyl methacrylate,8 wt. %, methyl acrylate 25 wt. %) | 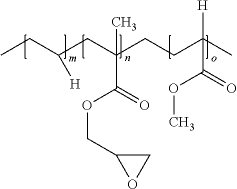 | melt index 6 g/10 min (190° C./ 2.16 kg) |
| 36604-80-5 | 2-Propenoic acid, 2- methyl-, oxiranylmethyl ester, polymer with ethene and ethenyl acetate | 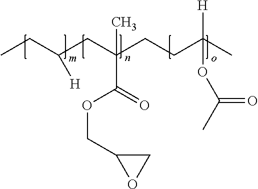 | |
| 58353-15-4 | 2-Propenoic acid, 2- methyl-, polymer with ethenylbenzene and oxiranylmethyl 2- methyl-2-propenoate | 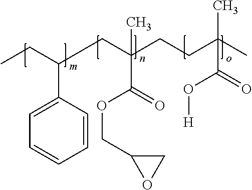 | |
| 70939-77-4 | Poly(tert-butyl methacrylate-co- glycidyl methacrylate) | 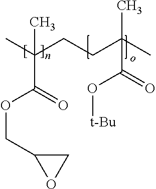 | |

1. A method comprising:
extruding a composition comprising a polymer and metal oxide through a twin screw extruder to form an extrudate;
milling the extrudate to form organic/inorganic composite particles,
wherein the metal oxide particles are dispersed throughout the organic/inorganic composite particles.

2. The method according to claim 1, wherein the milling step comprises jet milling.

3. The method according to claim 1 wherein the polymer comprises a reactive functional group.

4. The method according to claim 2 wherein the polymer comprises a reactive functional group.

5. The method according to claim 3 wherein the reactive functional group is a maleic anhydride group.

6. The method according to claim 4 wherein the reactive functional group is a maleic anhydride group.

7. The method according to claim 5 wherein the polymer comprises styrene maleic anhydride copolymer (SMA) or polypropylene-graft-maleic anhydride (PPMA).

8. The method according to claim 6 wherein the polymer comprises styrene maleic anhydride copolymer (SMA) or polypropylene-graft-maleic anhydride (PPMA).

9. The method according to claim 7, wherein the particles have an average diameter less than 10 μm.

10. The method according to claim 8, wherein the particles have an average diameter less than 10 μm.

11. The method according to claim 2, wherein the particles have an average diameter less than 5 μm.

12. The method according to claim 2 wherein the particles have an average diameter of between 1 μm and 10 μm.

13. The method according to claim 1 further comprising conjugating a biomolecule to the organic/inorganic composite particles.

14. An organic/inorganic composite comprising:
a thermoplastic polymer comprising a maleic anhydride group and iron oxide,
wherein the iron oxide is dispersed throughout the polymer,
wherein the composite has a Young's modulus greater than 1 gPa.

15. The organic/inorganic composite of claim 14 wherein the composite has a hardness of 0.08 gPa or greater.

16. The organic/inorganic composite of claim 14 wherein the composite comprises a Young's modulus of between 1 gPa and 7 gPa.

17. The organic/inorganic composite of claim 14, wherein the composite is in the form of particles less than 10 μm in average diameter.

18. The organic/inorganic composite of claim 14, wherein the composite is in the form of particles less than 5 μm in average diameter.

19. The organic/inorganic composite of claim 14 further comprising conjugating a biomolecule to the organic/inorganic composite particles.

20. The method according of claim 14 further comprising conjugating a plurality of biomolecules to the organic/inorganic composite particles.
